(19) United States
(12) Patent Application Publication
(10) Pub. No.: US 2025/0269155 A1
(43) Pub. Date: Aug. 28, 2025

(54) BALLOON FOR BALLOON CATHETER AND BALLOON CATHETER INCLUDING SAME

(71) Applicant: KANEKA CORPORATION, Osaka (JP)

(72) Inventors: Yoshinori Nakano, Osaka (JP); Kazuki Matsufuji, Osaka (JP); Masahiro Kojima, Osaka (JP); Masato Tsueda, Osaka (JP)

(73) Assignee: KANEKA CORPORATION, Osaka (JP)

(21) Appl. No.: 19/207,734

(22) Filed: May 14, 2025

Related U.S. Application Data
(63) Continuation of application No. PCT/JP2023/040823, filed on Nov. 13, 2023.

(30) Foreign Application Priority Data
Nov. 17, 2022 (JP) 2022-184269

(51) Int. Cl. A61M 25/10

(52) U.S. Cl. CPC A61M 25/104; A61M 2025/1075; A61M 2025/1088

(57) ABSTRACT

A balloon for a balloon catheter that, when inflated at a stenosed site, can easily contact the inner wall of the stenosis to improve the ability to dilate the stenosis and prevent displacement of the balloon in the longitudinal axis direction, thereby enhancing safety, is provided. The balloon includes a first layer disposed over the entire 360° in the circumferential direction and a second layer having a Shore D hardness higher than that of the first layer. The balloon includes, in a cross-section perpendicular to the longitudinal axis direction, a first part including the first layer where the second layer is not disposed on an outer side of the first layer in the radial direction, and a second part including the first layer and the second layer where the second layer is disposed on the outer side of the first layer in the radial direction.

### TECHNICAL FIELD

One or more embodiments of the present invention relate to a balloon for a balloon catheter and a balloon catheter including the same.

### BACKGROUND

Diseases such as angina pectoris and myocardial infarction are caused by the formation of stenosis in the inner wall of a blood vessel. One treatment method for these diseases is angioplasty, such as percutaneous transluminal coronary angioplasty (PTCA) or percutaneous transluminal angioplasty (PTA), in which a balloon catheter is used to dilate the stenosed site. Angioplasty is a minimally invasive therapy that does not require thoracotomy like bypass surgery, and it is widely performed.

In angioplasty, the distal end of a balloon provided at a distal portion of a balloon catheter is inserted from a puncture site such as the femoral artery or the brachial artery, and the balloon is delivered through a vascular lumen to a lesion site by manipulation from the proximal side of the balloon catheter. Accordingly, balloon catheters having a balloon with improved ease of insertion, or a balloon provided with protrusions capable of dilating the stenosed site, have been developed. For example, Patent document 1 discloses a balloon catheter in which a balloon formed of a single material over the entire balloon is reduced in diameter to improve ease of insertion, and Patent document 2 discloses a balloon catheter including protrusions having greater rigidity than the balloon wall and configured to perform a dilating function.

### PATENT DOCUMENT

Patent document 1: JP 2014-155657 A
Patent document 2: US 2016/0128718 A1

However, in the above conventional balloon catheter, when the balloon is inflated in a region having a complex lumen shape such as a stenosed site after being delivered to the lesion site, there have been issues in that the balloon may fail to sufficiently contact the inner wall of the lesion, making it difficult to properly dilate the stenosis, or that displacement in the longitudinal axis direction of the balloon may occur, leading to reduced safety of the treatment.

### SUMMARY

In view of the above circumstances, a balloon for a balloon catheter which, when inflated at a stenosed site, is more likely to come into contact with the inner wall of the stenosis, thereby improving the ability to dilate the stenosis, and which prevents displacement of the balloon in the longitudinal axis direction, thereby enhancing safety; and a balloon catheter comprising such a balloon, are provided.

A balloon for a balloon catheter according to one or more embodiments of the present invention, which has addressed the above, is as follows.

[1] A balloon for a balloon catheter having a longitudinal axis direction, a radial direction, and a circumferential direction, comprising: a first layer; and a second layer composed of a material having a Shore D hardness higher than that of the first layer, the first layer disposed over the entire 360° in the circumferential direction; the second layer located on an outer side of the first layer in the radial direction; the balloon having, in a cross-section perpendicular to the longitudinal axis direction, at least one first part composed only of the first layer and at least one second part composed of the first layer and the second layer; and the first part being disposed in the circumferential direction within a range of 90° or less out of the 360° in the circumferential direction.

[2] The balloon for a balloon catheter according to [1], wherein the first part extends in the longitudinal axis direction.

[3] The balloon for a balloon catheter according to [1] or [2], wherein the first part comprises a plurality of first parts and the second part comprises a plurality of second parts, wherein the first parts are disposed apart from each other in the circumferential direction such that an outer peripheral length in the circumferential direction per one of the second parts is twice or more an outer peripheral length in the circumferential direction per one of the first parts.

[4] The balloon for a balloon catheter according to any one of [1] to [3], wherein the second part having a thickness T1 of the first layer in the radial direction; and a thickness T2 of the second layer in the radial direction; and a total thickness T defined as a sum of T1 and T2, the balloon further comprising: a portion P1 in which T1 is more than 50% of T; and a portion P2 in which T2 is more than 50% of T.

[5] The balloon for a balloon catheter according to [4], further comprising, in the second part: a first-layer-rich portion in which an outer peripheral length in the circumferential direction of the portion P1 is 15% or more of the outer peripheral length in the circumferential direction per one of the second parts; and two or more second-layer-rich portions, each having an outer peripheral length in the circumferential direction of the portion P2 that is 15% or more of the outer peripheral length in the circumferential direction per one of the second parts, wherein the first-layer-rich portion is sandwiched between the second-layer-rich portions in the circumferential direction.

One or more embodiments of the present invention also provide the following.

[6] A balloon catheter comprising the balloon for a balloon catheter according to any one of [1] to [5].

According to the above-described balloon for a balloon catheter and the balloon catheter including the same, when the balloon is inflated at a stenosed site, the balloon is more likely to come into contact with the inner wall of the stenosis, making it possible to improve the ability to dilate the stenosis and to prevent displacement of the balloon in the longitudinal axis direction, thereby enhancing safety. In addition, since the rigidity of the balloon can be made equal to or greater than a predetermined level, it is possible to provide a balloon for a balloon catheter and a balloon catheter including the same with improved pressure resistance.

### BRIEF DESCRIPTION OF THE DRAWINGS

FIG. 1 is a side view of a balloon catheter according to one or more embodiments of the present invention.

FIG. 2 is a cross-sectional view taken along line II-II of the balloon catheter shown in FIG. 1.

FIG. 3 is a plan view of a distal end portion of the balloon catheter shown in FIG. 1.

FIG. 4 is a variation of the cross-sectional view taken along line II-II.

FIG. 5 is an enlarged view of the second part in the cross-sectional view shown in FIG. 4.

FIG. 6 is a perspective view of a parison before biaxial stretching, according to one or more embodiments of the present invention.

FIG. 7 is a cross-sectional view taken along line VII-VII of the parison shown in FIG. 6.

FIG. 8 is a cross-sectional view perpendicular to the longitudinal axis direction of a parison mold used for manufacturing the parison shown in FIG. 7.

FIG. 9 is a cross-sectional view perpendicular to the longitudinal axis direction after removal of a protrusion part of the parison shown in FIG. 7.

FIG. 10 is a variation of the cross-sectional view taken along line VII-VII.

FIG. 11 is a cross-sectional view perpendicular to the longitudinal axis direction of a parison mold used for manufacturing the parison shown in FIG. 10.

FIG. 12 is a cross-sectional view perpendicular to the longitudinal axis direction after removal of a protrusion part of the parison shown in FIG. 10.

FIG. 13 is a longitudinal cross-sectional view of a mold according to one or more embodiments of the present invention.

FIG. 14 is a cross-sectional view taken along line XIV-XIV in FIG. 13.

### DETAILED DESCRIPTION

Hereinafter, one or more embodiments of the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of one or more embodiments of the present invention, the dimensions may differ from the actual dimensions in some cases.

### 1. Balloon for Balloon Catheter

A balloon for a balloon catheter according to one or more embodiments of the present invention has a longitudinal axis direction, a radial direction, and a circumferential direction; and has a first layer and a second layer composed of a material having a Shore D hardness higher than that of the first layer. The first layer is disposed over the entire 360° in the circumferential direction; the second layer is located on an outer side of the first layer in the radial direction. In a cross-section perpendicular to the longitudinal axis direction, the balloon has at least one first part composed only of the first layer, and at least one second part composed of the first layer and the second layer; and the first part is disposed in the circumferential direction within a range of 90° or less out of the 360° in the circumferential direction.

In order to dilate a stenosed site using a balloon catheter, it is necessary to insert a balloon provided at a distal end portion of the balloon catheter into a vascular lumen, deliver the balloon to the stenosed site, inflate the balloon, and press the outer wall of the balloon against the inner wall of the stenosis. In this case, the outer wall of the balloon may not be able to sufficiently contact the inner wall of the stenosis. However, according to the above-described balloon for a balloon catheter, at least one first part composed only of the first layer having a lower Shore D hardness is provided in a cross-section perpendicular to the longitudinal axis direction, allowing the flexible first part to function as a buffer body capable of stretching and contracting. As a result, the outer wall of the balloon can more easily conform to the shape of the vascular lumen at the stenosed site, making it possible to improve the ability of the balloon to dilate the stenosis. In addition, since the outer wall of the balloon can sufficiently contact the inner wall of the stenosis, displacement of the balloon in the longitudinal axis direction can be prevented. This makes it possible to prevent the balloon from acting on parts of the vascular wall other than the treatment target site, thereby enhancing safety. Furthermore, the second layer having a higher Shore D hardness is located on an outer side of the first layer in the radial direction, and the range in which the first part composed only of the first layer is disposed in the circumferential direction is 90 or less out of the 360° in the circumferential direction. Therefore, the range in which the second layer is present on the outer wall of the balloon can be set to at least 270° in the circumferential direction. Accordingly, the higher Shore D hardness of the second layer allows the rigidity of the balloon to be equal to or greater than a predetermined level, which facilitates improved pressure resistance and enhanced efficiency in dilating the stenosis.

In the present specification, the balloon for a balloon catheter may sometimes be simply referred to as “balloon.”

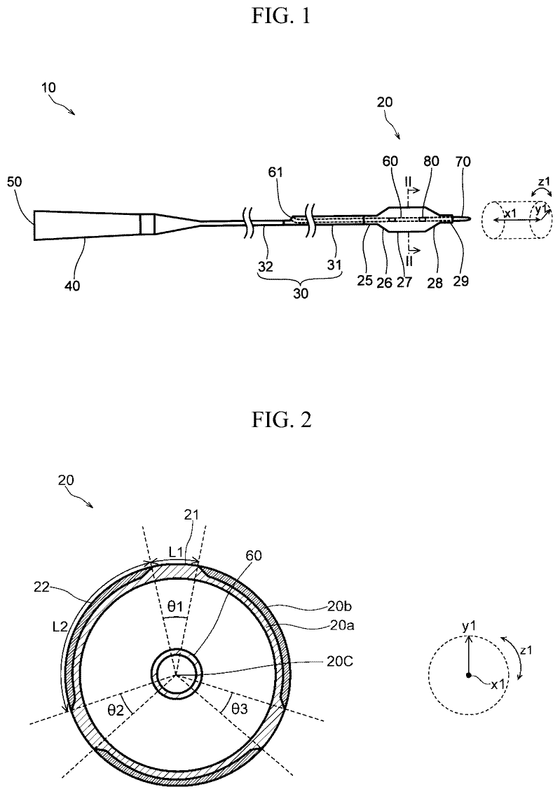

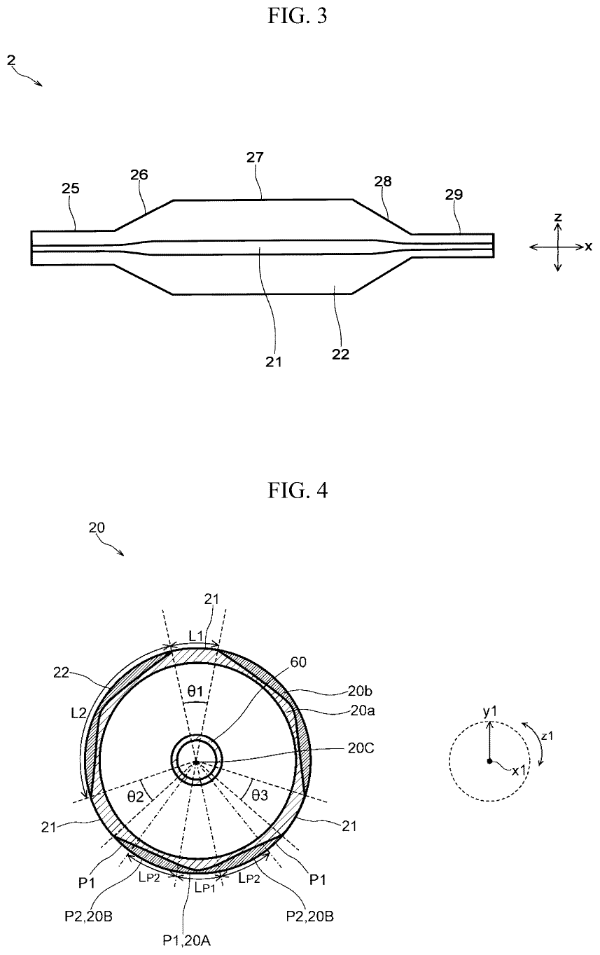

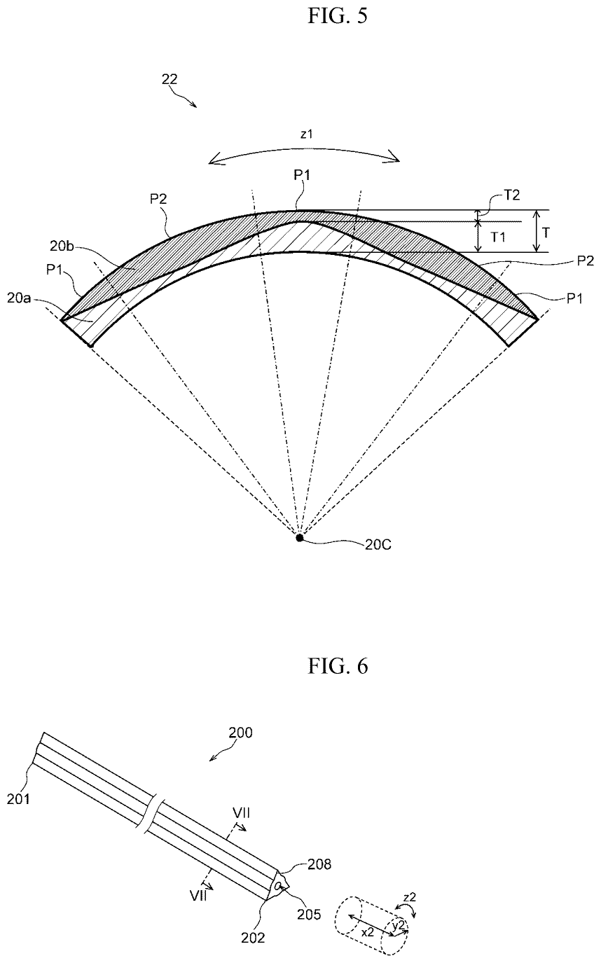

Hereinafter, a balloon for a balloon catheter according to one or more embodiments of the present invention will be described with reference to FIGS. 1 to 5. FIG. 1 is a side view of a balloon catheter according to one or more embodiments of the present invention. FIG. 2 is a cross-sectional view taken along line II-II of the balloon catheter shown in FIG. 1. In FIG. 2, the boundary between the regions in which the first part and the second part are disposed is indicated by a dotted line. FIG. 3 is a plan view of a distal end portion of the balloon catheter shown in FIG. 1, and shows the balloon as viewed from the side where the first part is present. FIG. 4 is a variation of the cross-sectional view taken along line II-II. In FIG. 4, the boundary between the regions in which the first part and the second part are disposed is indicated by a dotted line, and the boundary between the regions in which the first-layer-rich portion and the second-layer-rich portions are disposed is indicated by a chain double-dashed line. FIG. 5 is an enlarged view of the second part in the cross-sectional view shown in FIG. 4, in which the boundary between the regions in which the first-layer-rich portion and the second-layer-rich portions are disposed is indicated by a chain double-dashed line. The inner shaft is omitted in FIG. 5.

As shown in FIG. 1, a balloon 20 is provided at a distal portion of a balloon catheter 10. The balloon 20 is connected to a distal end portion of a shaft 30, and the balloon 20 can be inflated by introducing a fluid through a lumen of the shaft 30 and can be deflated by discharging the fluid. In order to control inflation and deflation of the balloon 20, a fluid may be introduced or discharged using an indeflator (balloon pressurizer). The fluid may be a pressurized fluid that is pressurized by a pump or the like. Details of the balloon catheter 10 will be described in the section “2. Balloon catheter.”

The balloon 20 has a longitudinal axis direction x1, a radial direction y1 that connects the centroid 20C of the outer edge of the balloon 20 and a point on the outer edge in a cross-section perpendicular to the longitudinal axis direction x1, and a circumferential direction z1 that extends along the outer edge of the balloon 20 in the same cross-section. In the present specification, the direction toward the operator's side in the longitudinal axis direction x1 is referred to as the proximal side, and the opposite side, i.e., the direction toward the patient, is referred to as the distal side.

Members or portions other than the balloon 20 each have a longitudinal axis direction, a radial direction, and a circumferential direction. These directions may be the same as, or different from, the longitudinal axis direction x1, the radial direction y1, and the circumferential direction z1 of the balloon 20. However, for ease of understanding, in the present specification, all members and portions are described as having the same longitudinal axis direction, radial direction, and circumferential direction as the longitudinal axis direction x1, radial direction y1, and circumferential direction z1 of the balloon 20.

As shown in FIG. 2, the balloon 20 includes a first layer 20a and a second layer 20b composed of a material having a Shore D hardness higher than that of the first layer 20a. The first layer 20a is disposed over the entire 360° in the circumferential direction z1. The second layer 20b is located on an outer side of the first layer 20a in the radial direction y1. In a cross-section perpendicular to the longitudinal axis direction x1, the balloon 20 has at least one first part 21 composed only of the first layer 20a and at least one second part 22 composed of the first layer 20a and the second layer 20b.

Since the first layer 20a having a lower Shore D hardness is continuously disposed over the entire 360° in the circumferential direction z1, the flexibility of the balloon 20 can be improved. In a cross-section perpendicular to the longitudinal axis direction x1, at least one first part 21 composed only of the first layer 20a having a lower Shore D hardness is provided, allowing the flexible first part 21 to function as a buffer body capable of stretching and contracting. As a result, the outer wall of the balloon 20 can more easily conform to the shape of the vascular lumen at the stenosed site, making it possible to improve the ability of the balloon 20 to dilate the stenosis. In addition, since the outer wall of the balloon 20 can sufficiently contact the inner wall of the stenosis, displacement of the balloon 20 in the longitudinal axis direction x1 can be prevented. This makes it possible to prevent the balloon 20 from acting on parts of the vascular wall other than the treatment target site, thereby enhancing safety.

In the circumferential direction z1, the range in which the first part 21 is disposed is 90° or less out of the 360° in the circumferential direction z1. Therefore, the range in which the second part 22, in which the second layer 20b having a higher Shore D hardness is located on an outer side in the radial direction y1 and the first layer 20a having a lower Shore D hardness is located on an inner side in the radial direction y1, is present in the circumferential direction z1 can be set to at least 270° out of the 360° in the circumferential direction z1. As a result, the second layer 20b having a higher Shore D hardness can be disposed on the outer wall of the balloon 20 over at least 270° out of the 360° in the circumferential direction z1, thereby enabling improved pressure resistance and enhanced efficiency in dilating the stenosis with the second layer 20b.

In the circumferential direction z1, the range in which the first part 21 is disposed may be 80° or less out of the 360° in the circumferential direction z1, 70° or less, or 60° or less. When the upper limit of the range in which the first part 21 is disposed is as described above, a sufficient range for the second part 22 to be disposed can be ensured. As a result, the second part 22 including the second layer 20b having a higher Shore D hardness can contribute to improved pressure resistance and enhanced efficiency in dilating the stenosis. The range in which the first part 21 is disposed in the circumferential direction z1 may be 5° or more, 10° or more, 15° or more, 20° or more, or 30° or more. When the lower limit of the range in which the first part 21 is disposed is as described above, the first part 21 can function as a buffer body, allowing the outer wall of the balloon 20 to more easily conform to the shape of the vascular lumen at the stenosed site. When a plurality of first parts 21 are provided in the circumferential direction z1, the respective ranges in which the first parts 21 are disposed in the circumferential direction z1 may be the same or different.

At least one first part 21 is provided in the circumferential direction z1. The number of first parts 21 in the circumferential direction z1 may be two or more, three or more, or four or more. The number may be ten or less, eight or less, or six or less. One first part 21 refers to a portion in which the first part 21, that is, a portion composed only of the first layer 20a, continuously exists in the circumferential direction z1. A second part 22 is present between two adjacent first parts 21 in the circumferential direction z1. Since the portions other than the first parts 21 in the balloon 20 are the second parts 22, the number of second parts 22 provided in the circumferential direction z1 is the same as the number of the first parts 21.

As shown in FIG. 2, the range in which the first part 21 is disposed can be defined, in a cross-section perpendicular to the longitudinal axis direction x1, as the smaller angle formed by two line segments connecting the centroid 20C of the outer edge of the balloon 20 and both ends of the first part 21 on the outer edge of the balloon 20. When one first part 21 is provided in the circumferential direction z1, the range in which the first part 21 is disposed corresponds to the above-mentioned angle. When a plurality of first parts 21 are provided in the circumferential direction z1, the above-mentioned angle is determined for each of the first parts 21, and the total sum of these angles represents the overall range in which the first parts 21 are disposed in the circumferential direction z1. For example, as shown in FIG. 2, when three first parts 21 are provided in the circumferential direction z1, the range in which the first parts 21 are disposed in the circumferential direction z1 is the sum of angles θ1, θ2, and θ3. Therefore, when a plurality of first parts 21 are provided in the circumferential direction z1, the balloon 20 has the first parts 21 such that the total sum of the angles of the respective first parts 21 is 90° or less. The greater the number of first parts 21 provided in the circumferential direction z1, the smaller the angle of each individual first part 21, that is, the smaller the range in which each individual first part 21 is disposed.

As shown in FIG. 2, the balloon 20 may be composed only of the first layer 20a and the second layer 20b. Alternatively, although not shown in the figures, the balloon 20 may have one or more layers other than the first layer 20a and the second layer 20b. When the balloon 20 includes layers other than the first layer 20a and the second layer 20b, the balloon 20 may have a configuration in which a first balloon film composed only of the first layer 20a and the second layer 20b includes the first part 21 and the second part 22, and a second balloon film or a third balloon film is disposed on the inner side or the outer side in the radial direction y1 of the first balloon film. Even in such a configuration, as long as the first balloon film includes the first part 21 and the second part 22 within the above-defined ranges, the balloon having the multiple-film configuration is included in the balloon 20 according to one or more embodiments of the present invention.

The Shore D hardness of the first layer 20a may be 20 or more, 25 or more, 30 or more, 35 or more, or 40 or more. The Shore D hardness of the first layer 20a may be 70 or less, 65 or less, 60 or less, or 55 or less. The Shore D hardness of the second layer 20b may be more than 70, 72 or more, 74 or more, or 75 or more. The Shore D hardness of the second layer 20b may also be 90 or less, 85 or less, or 80 or less. When the Shore D hardness of the first layer 20a is within the above range, it is possible to realize the buffer function of the first part 21 and to contribute to the improvement in the flexibility of the balloon 20. When the Shore D hardness of the second layer 20b is within the above range, it is possible to contribute to the improvement in pressure resistance and enhanced efficiency in dilating the stenosis by the second layer 20b.

The Shore D hardness can be measured, for example, using a Type D durometer in accordance with JIS K6253-2:2012. The respective Shore D hardness values of the first layer 20a and the second layer 20b may be values of the materials before being formed into the balloon 20.

As the material of the second layer 20b, polyamide resins such as nylon 11 and nylon 12; polyester resins such as polyethylene terephthalate and polybutylene terephthalate; and polyurethane resins may be used. As the material of the first layer 20a, a thermoplastic elastomer may be used from the viewpoint of having a lower Shore D hardness, and for example, a polyamide elastomer such as a polyether block amide copolymer may be used.

As shown in FIG. 1, the balloon 20 may have a proximal end and a distal end in the longitudinal axis direction x1, and include a straight tubular part 27, a proximal tapered part 26 located on the proximal side of the straight tubular part 27, a proximal sleeve part 25 located on the proximal side of the proximal tapered part 26, a distal tapered part 28 located on the distal side of the straight tubular part 27, and a distal sleeve part 29 located on the distal side of the distal tapered part 28. The straight tubular part 27 may be substantially cylindrical with approximately the same diameter in the longitudinal axis direction x1, but it may have different diameters along the longitudinal axis direction x1. The proximal tapered part 26 and the distal tapered part 28 may be formed in a substantially conical or frustoconical shape that gradually decreases in diameter as it extends away from the straight tubular part 27. By having the maximum diameter in the straight tubular part 27, the straight tubular part 27 can more easily come into sufficient contact with the inner wall of the stenosed site during inflation of the balloon 20, thereby facilitating treatment such as dilation of the stenosis. In addition, since the proximal tapered part 26 and the distal tapered part 28 are reduced in diameter, the outer diameters of the proximal and distal end portions of the balloon 20 become smaller when the balloon 20 is deflated. As a result, the step between the shaft 30 and the balloon 20 can be reduced, making it easier to insert the balloon 20 into a body cavity.

The proximal tapered part 26, the straight tubular part 27, and the distal tapered part 28 may be portions that expand when fluid is introduced into the balloon 20, whereas the proximal sleeve part 25 and the distal sleeve part 29 may be portions that do not expand. This allows at least a part of the proximal sleeve part 25 to be fixed to a distal end portion of the shaft 30, and at least a part of the distal sleeve part 29 to be fixed to an inner shaft 60, which will be described later.

As shown in FIG. 3, the first part 21 may be extended in the longitudinal axis direction x1. This facilitates radial expansion and contraction of the balloon 20 by allowing the first part 21 to function as a buffer body, thereby making it easier for the balloon 20 to conform to the inner wall of the stenosed site.

The first part 21 may be provided over the entire length of the balloon 20 in the longitudinal axis direction x1, or may be provided only over a portion thereof. When the first part 21 is provided over the entire length in the longitudinal axis direction x1 of the balloon 20, the balloon 20 can expand and contract in the radial direction y1 along its entire length, and the overall shape of the balloon 20 in the longitudinal axis direction x1 can be controlled. Alternatively, when the first part 21 is provided only over a portion of the balloon 20 in the longitudinal axis direction x1, the portion where the first part 21 is provided can expand and contract in the radial direction y1, while the portion where the first part 21 is not provided is less expandable and contractible in the radial direction y1. This allows the desired portion of the balloon 20 to be selectively expanded and contracted in accordance with the inner diameter shape of the stenosed site to be treated.

For example, the first part 21 can be provided in the straight tubular part 27. By providing the first part 21 in the straight tubular part 27, the portion of the balloon 20 that inflates the most can be configured to expand and contract in the radial direction y1. In this case, the first part 21 may or may not be provided in the tapered parts or the sleeve parts. The first part 21 may extend over the entire region from the proximal end to the distal end of the straight tubular part 27 in the longitudinal axis direction x1, or it may extend over only a portion of that region, with the remainder not provided with the first part 21.

Alternatively, the first part 21 can be provided from the straight tubular part 27 to the distal tapered part 28. By providing the first part 21 from the straight tubular part 27 to the distal tapered part 28, the distal side of the balloon 20 can be configured to expand and contract in the radial direction y1. The first part 21 can also be provided from the straight tubular part 27 to the proximal tapered part 26. By providing the first part 21 from the straight tubular part 27 to the proximal tapered part 26, the proximal side of the balloon 20 can be configured to expand and contract in the radial direction y1.

The first part 21 may extend over the entire region from the proximal end to the distal end of the tapered part in the longitudinal axis direction x1, or may extend only over a portion of the tapered part, with the remainder not provided with the first part 21. When the first part 21 extends over a part of the tapered part in the longitudinal axis direction x1, it may be provided on the distal side and not on the proximal side with respect to the midpoint of the proximal tapered part 26, and may be provided on the proximal side and not on the distal side with respect to the midpoint of the distal tapered part 28. That is, the first part 21 may be provided on the side adjacent to the straight tubular part 27 and not on the side adjacent to the sleeve part. With such a configuration, the first part 21 can be provided in the portion of the balloon 20 that inflates more significantly, thereby facilitating expansion and contraction of the balloon 20.

The first part 21 may not be provided in the proximal sleeve part 25 and/or the distal sleeve part 29. This is because the sleeve parts are portions that do not inflate even when fluid is introduced into the balloon 20, and therefore, there is no need for the balloon 20 to expand and contract in the radial direction y1 by means of the first part 21 in those regions. Alternatively, the first part 21 may also be provided in the sleeve parts.

The first part 21 may extend linearly along the longitudinal axis direction x1, or may extend in a helical shape. The first part 21 may also be provided continuously in the longitudinal axis direction x1, or may be provided discontinuously.

As shown in FIG. 3, the second part 22 may also be extended in the longitudinal axis direction x1. Since the portion of the balloon 20 other than the first part 21 may be the second part 22, when the first part 21 extends in the longitudinal axis direction x1, it becomes easier for the second part 22 to also extend in the longitudinal axis direction x1. By extending the second part 22 in the longitudinal axis direction x1, it becomes easier to improve the pressure resistance and enhance the ability of the second part 22 to dilate the stenosed site.

As shown in FIG. 2, in a cross-section perpendicular to the longitudinal axis direction x1, the balloon 20 may include a plurality of first parts 21 and a plurality of second parts 22. The first parts 21 may be disposed apart from each other in the circumferential direction z1 such that an outer peripheral length L2 in the circumferential direction z1 per one of the second parts 22 is twice or more an outer peripheral length L1 in the circumferential direction z1 per one of the first parts 21.

The first parts 21 may be disposed apart from each other in the circumferential direction z1 such that the outer peripheral length L2 in the circumferential direction z1 per one of the second parts 22 may be 2.5 times or more the outer peripheral length L1 in the circumferential direction z1 per one of the first parts 21, 3 times or more, or 4 times or more. Although the upper limit of the ratio of L2 to L1 is not particularly limited, it may be, for example, 50 times or less, 30 times or less, or 10 times or less.

By disposing the first parts 21 apart from each other in the circumferential direction z1 at a predetermined interval or more, the balloon 20 can more easily expand and contract in the circumferential direction z1 over the entire circumference, making it easier for the outer wall of the balloon 20 to conform to the shape of the lumen at the stenosed site. In addition, since the length of the second parts 22 in the circumferential direction z1 can be set to a predetermined value or more, it becomes easier to ensure the rigidity of the balloon 20, thereby facilitating improvements in pressure resistance and the ability of the second parts 22 to dilate the stenosed site.

As shown in FIGS. 4 and 5, the second part 22 has a thickness T1 of the first layer 20a in the radial direction y1; a thickness T2 of the second layer 20b in the radial direction y1; and a total thickness T defined as a sum of T1 and T2. The balloon 20 further comprises: a portion P1 in which T1 is more than 50% of T; and a portion P2 in which T2 is more than 50% of T. Since the total thickness T is defined as the sum of T1 and T2, the second part 22 is composed of either the portion P1 or the portion P2, excluding regions where T1 and T2 are exactly 50% of T. When the balloon 20 is composed only of the first layer 20a and the second layer 20b, that is, when the balloon 20 includes only the above-described first balloon film, the total thickness T corresponds to the overall thickness of the balloon 20.

By having the portions P1 and P2 in the second part 22, the balloon 20 can improve flexibility in the portion P1 due to the lower rigidity of the first layer 20a, and can improve strength and pressure resistance in the portion P2 due to the higher rigidity of the second layer 20b. In this way, in the second part 22, in which the high-rigidity second layer 20b is disposed on the outer surface of the balloon 20 and contributes to the incision of the stenosed site, both the higher-rigidity portion P2 and the lower-rigidity portion P1 can coexist, thereby enabling simultaneous achievement of these opposing effects.

The thickness of the balloon 20 may be 12 μm or more, 15 μm or more, or 20 μm or more, and 60 μm or less, 50 μm or less, or 40 μm or less. When the balloon 20 is composed only of the first layer 20a and the second layer 20b, the total thickness T may be within the above range.

The thickness of the first layer 20a in the first part 21 can be substantially equal to the thickness of the above-described balloon 20.

The thickness of the first layer 20a in the second part 22 can be less than the overall thickness of the balloon 20 and may be, for example, 2 μm or more, 3 μm or more, 4 μm or more, 5 μm or more, or 6 μm or more, and 55 μm or less, 50 μm or less, 40 μm or less, 30 μm or less, or 20 μm or less. The thickness of the second layer 20b in the second part 22 can also be less than the overall thickness of the balloon 20 and may be, for example, 2 μm or more, 3 μm or more, 4 μm or more, 5 μm or more, or 6 μm or more, and 55 μm or less, 50 μm or less, 40 μm or less, 30 μm or less, or 20 μm or less. When the portions P1 and P2 are to be provided in the second part 22, it is sufficient to vary the thicknesses of the first layer 20a and the second layer 20b within the above ranges.

At any position in the longitudinal axis direction x1, the variation rate of the wall thickness Tx of the balloon 20 at any position x in the circumferential direction z1 relative to the average wall thickness Ta of the balloon 20 (|Tx−Ta|/Ta)× 100 may be 15% or less, 10% or less, 8% or less, or 5% or less. This makes it possible to form the outer surface of the balloon 20 so as not to protrude outward in the radial direction y1, thereby enabling the outer wall of the balloon 20 to more easily conform to the shape of the lumen at the stenosed site. Although the ideal lower limit of the variation rate of the wall thickness Tx of the balloon 20 is 0%, it may be, in practice, 1% or more, 2% or more, or 3% or more.

The wall thickness of the balloon 20, the thickness T1 of the first layer 20a in the radial direction y1, and the thickness T2 of the second layer 20b in the radial direction y1 can be measured by observing a cross-section of the balloon 20 perpendicular to the longitudinal axis direction x1. For observation, for example, an optical microscope can be used, and the thickness can be obtained from the measured thickness on the resulting observation image and the magnification used in the observation.

The average wall thickness Ta of the balloon 20 can be obtained by measuring the wall thickness at 24 points spaced at 15° intervals over the full 360° in the circumferential direction z1 of the balloon 20 and calculating the average of those 24 values. These 24 points can be defined by drawing 24 radial lines from the centroid 20C of the outer edge of the balloon 20 to the outer edge such that the central angle (the smaller one) formed between adjacent lines is 15°. The number of measurement points for obtaining the average wall thickness Ta is not limited to 24 and may be smaller or greater; however, it may be 8 or more.

The thickness may be measured by observing the balloon 20 in its inflated state. Although the method for maintaining the balloon 20 in the inflated state is not particularly limited, examples include a method in which the inflated balloon 20 is embedded in a curable resin for observation, a cross-section perpendicular to the longitudinal axis direction x1 is exposed, and the cross-section is observed. Alternatively, the balloon 20 may be directly observed without being embedded in the curable resin, or the balloon 20 in a deflated state may be observed as long as the central angle can be determined.

The wall thickness Tx of the balloon 20 at any position x in the circumferential direction z1 is not limited to the measurement points used to determine the average wall thickness Ta, and can be obtained by observing the wall thickness at any position x in the circumferential direction z1 using the same method as described above.

The thickness T1 of the first layer 20a in the radial direction y1 and the thickness T2 of the second layer 20b in the radial direction y1 can also be measured in the same manner as the wall thickness of the balloon 20. Since the first layer 20a and the second layer 20b are formed from different resins, the boundary between the layers can be observed under a microscope, allowing the thickness of each layer to be determined.

As shown in FIG. 4, the balloon 20 further has, in the second part 22, a first-layer-rich portion 20A in which an outer peripheral length L_{P1} in the circumferential direction z1 of the portion P1 is 15% or more of the outer peripheral length L2 in the circumferential direction z1 per one of the second parts 22, and a second-layer-rich portion 20B in which an outer peripheral length L_{P2} in the circumferential direction z1 of the portion P2 is 15% or more of the outer peripheral length L2 in the circumferential direction per one of the second parts 22. In the circumferential direction z1, the first-layer-rich portion 20A may be sandwiched between the second-layer-rich portions 20B. It may also be preferable that the balloon 20 include at least one first-layer-rich portion 20A and two or more second-layer-rich portions 20B in the second part 22. Accordingly, the balloon 20 can be configured such that the flexible first-layer-rich portion 20A is sandwiched between the rigid second-layer-rich portions 20B within the second part 22, which occupies a wide range of at least 270° out of 360° in the circumferential direction z1. As a result, the flexible portions and rigid portions can be distributed throughout the entire circumferential direction z1 of the balloon 20, making it easier for the outer wall of the balloon 20 to conform to the shape of the lumen at the stenosed site.

It may be preferable that the outer peripheral length L_{P1} in the circumferential direction z1 of the portion P1 in the first-layer-rich portion 20A be 20% or more of the outer peripheral length L2 in the circumferential direction z1 per one of the second parts 22, and it may be 30% or more, and 70% or less, 60% or less, or 50% or less. It may be preferable that the outer peripheral length L_{P2} in the circumferential direction z1 of the portion P2 in each of the second-layer-rich portions 20B be 18% or more of the outer peripheral length L2 in the circumferential direction z1 per one of the second parts 22, and it may be 20% or more, and 70% or less, 60% or less, or 50% or less.

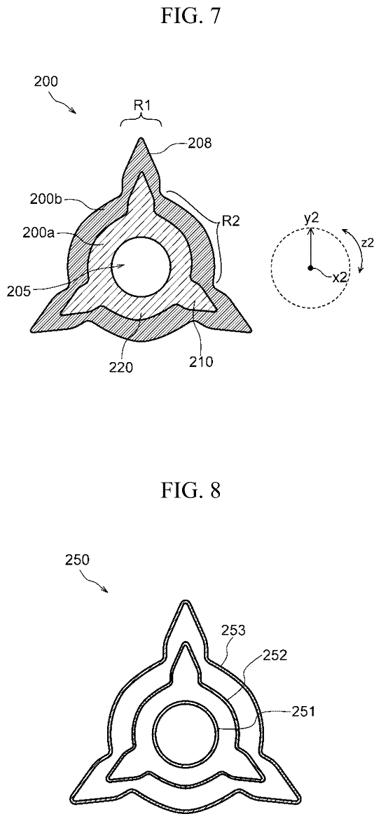

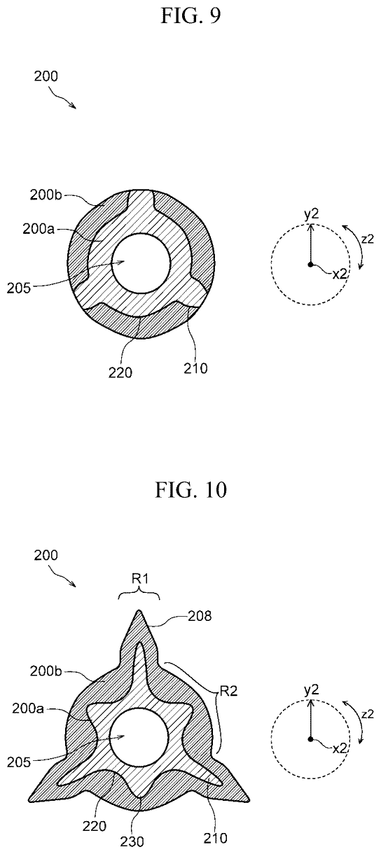

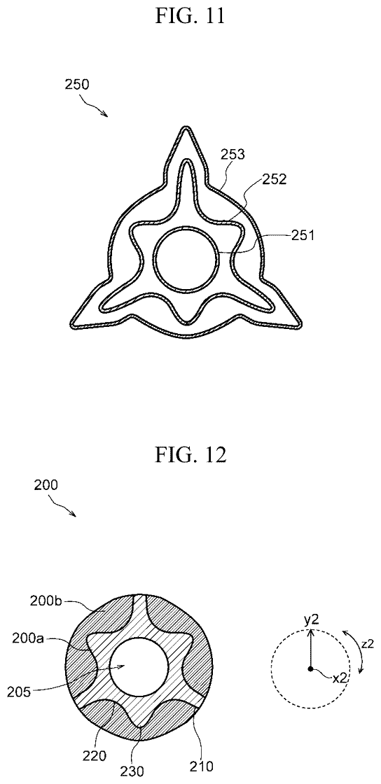

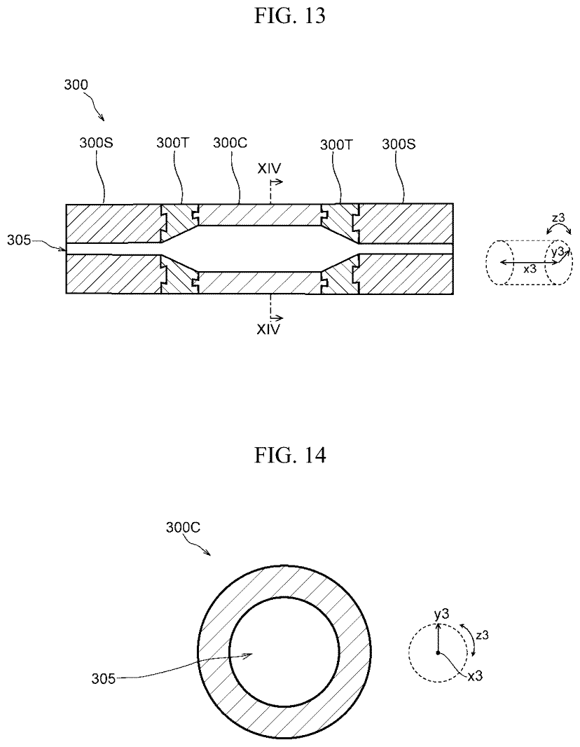

Next, a method for manufacturing the above-described balloon 20 according to one or more embodiments of the present invention will be described with reference to FIGS. 6 to 14. FIG. 6 is a perspective view of a parison before biaxial stretching, according to one or more embodiments of the present invention. FIG. 7 is a cross-sectional view taken along line VII-VII of the parison shown in FIG. 6, and represents a cross-sectional view of a parison used for manufacturing a balloon having the cross-section shown in FIG. 2. FIG. 8 is a cross-sectional view perpendicular to the longitudinal axis direction of a parison mold used for manufacturing the parison shown in FIG. 7. FIG. 9 is a cross-sectional view perpendicular to the longitudinal axis direction after removal of a protrusion part of the parison shown in FIG. 7. FIG. 10 is a variation of the cross-sectional view taken along line VII-VII, and represents a cross-sectional view of a parison used for manufacturing a balloon having the cross-section shown in FIG. 4. FIG. 11 is a cross-sectional view perpendicular to the longitudinal axis direction of a parison mold used for manufacturing the parison shown in FIG. 10. FIG. 12 is a cross-sectional view perpendicular to the longitudinal axis direction after removal of a protrusion part of the parison shown in FIG. 10. FIG. 13 is a longitudinal cross-sectional view of a mold used during biaxial stretching of the parison, according to one or more embodiments of the present invention. FIG. 14 is a cross-sectional view taken along line XIV-XIV in FIG. 13.

First, a parison 200 is prepared. The parison 200 is made of a resin and is a tubular member having a lumen 205, as shown in FIG. 6. The parison 200 has a first end 201 and a second end 202, and extends in a longitudinal axis direction x2 from the first end 201 to the second end 202. Similar to the balloon 20, the parison 200 has a radial direction y2 and a circumferential direction z2.

As shown in FIG. 7, the parison 200 includes a second layer 200b and a first layer 200a that is composed of a material having a lower Shore D hardness than that of the second layer 200b. The first layer 200a and the second layer 200b may be continuous over the entire circumferential direction z2. The materials forming the first layer 200a and the second layer 200b, as well as their Shore D hardness values, can be referred to in the descriptions of the resins forming the first layer 20a and the second layer 20b of the above-described balloon 20, and the descriptions of their Shore D hardness.

As shown in FIGS. 6 and 7, the parison 200 may include a protruding region R1 including a protrusion part 208 that protrudes outward in the radial direction y2 and extends in the longitudinal axis direction x2, and a non-protruding region R2 other than the protruding region R1. In a cross-section perpendicular to the longitudinal axis direction x2, the outer edge of the first layer 200a may protrude along the outer edge of the protrusion part 208. That is, it may be preferable that the first layer 200a include a thin portion 220 in the non-protruding region R2 and a thick portion 210, which is thicker than the thin portion 220, in the protruding region R1.

As shown in FIG. 7, a plurality of protrusion parts 208 may be provided in the circumferential direction z2. Although not shown in the figures, a single protrusion part 208 may alternatively be provided in the circumferential direction z2. When a plurality of protrusion parts 208 are provided in the circumferential direction z2, they may be spaced apart from each other in the circumferential direction z2, or may be arranged at equal intervals.

Such a parison 200 can be manufactured, for example, by extruding a resin using a parison mold 250 as shown in FIG. 8. As shown in FIG. 8, the parison mold 250 includes a first tubular member 251, a second tubular member 252, and a third tubular member 253. The first tubular member 251 has a cylindrical shape configured to form the lumen 205 of the parison 200. The second tubular member 252 has a cylindrical shape with a protrusion configured to form the thick portion 210 and the thin portion 220 of the first layer 200a. The third tubular member 253 may have a cylindrical shape with a protrusion configured to form the protrusion part 208. With this configuration, a resin for forming the first layer 200a is introduced into the space between the outer surface of the first tubular member 251 and the inner surface of the second tubular member 252, and a resin for forming the second layer 200b is introduced into the space between the outer surface of the second tubular member 252 and the inner surface of the third tubular member 253. Through extrusion molding, the parison 200 can be produced that includes the lumen 205, the first layer 200a, and the second layer 200b, wherein the first layer 200a has the thick portion 210 in the protruding region R1 and the thin portion 220 in the non-protruding region R2.

The material constituting the parison mold 250 may be a metal, and the metal may be iron, copper, aluminum, or an alloy thereof. For example, stainless steel is a suitable alloy of iron, brass is a suitable alloy of copper, and duralumin is a suitable alloy of aluminum. From the standpoint of having sufficient strength and ease of processing, the parison mold 250 may be made of stainless steel.

Prior to stretching the parison 200, the protrusion part 208 may be cut along the longitudinal axis direction x2 to form a parison 200 as shown in FIG. 9, in which a portion of the first layer 200a is exposed outward in the radial direction y2 in a cross-section perpendicular to the longitudinal axis direction x2. By stretching such a parison 200, the portion of the first layer 200a of the parison 200 having the thick portion 210 forms the first part 21, and the portion of the first layer 200a of the parison 200 having the thin portion 220 forms the second part 22, thereby enabling production of the balloon 20 having a cross-section as shown in FIG. 2.

Alternatively, the parison 200 may have a configuration as shown in FIG. 10. That is, in a cross-section perpendicular to the longitudinal axis direction x2, the first layer 200a has a medium-thickness portion 230, which has a thickness greater than that of the thin portion 220 and less than that of the thick portion 210, in the non-protruding region R2. The parison 200 may be configured such that the thin portion 220 is located between the thick portion 210 and the medium-thickness portion 230 in the circumferential direction z2.

The parison 200 as shown in FIG. 10 can be manufactured, for example, by extrusion molding a resin using the parison mold 250 as shown in FIG. 11. In the parison mold 250 shown in FIG. 11, the second tubular member 252 may have a cylindrical shape with a protrusion in the portion that forms the protruding region R1 and a low protrusion with a height lower than that of the protrusion in the portion that forms the non-protruding region R2, so that the thick portion 210, the thin portion 220, and the medium-thickness portion 230 of the first layer 200a can be formed. In this way, by a method similar to that described above, it is possible to manufacture the parison 200 having the lumen 205, the first layer 200a, and the second layer 200b, in which the first layer 200a has the thick portion 210 in the protruding region R1 and has the thin portion 220 and the medium-thickness portion 230 in the non-protruding region R2.

Prior to stretching the parison 200 shown in FIG. 10, the protrusion part 208 is cut along the longitudinal axis direction x2, thereby forming the parison 200 shown in FIG. 12, which has a portion where the first layer 200a is exposed outward in the radial direction y2 in a cross-section perpendicular to the longitudinal axis direction x2. By stretching the parison 200 in this state, the portion of the first layer 200a of the parison 200 that includes the thick portion 210 forms the first part 21, and the portion of the first layer 200a of the parison 200 that includes the thin portion 220 and the medium-thickness portion 230 forms the second part 22, thereby manufacturing the balloon 20 having the cross-section shown in FIG. 4.

By stretching the parison 200 after the protrusion part 208 has been removed, it is possible to manufacture the balloon 20 that includes the first part 21 composed only of the first layer 20a of the parison 200 and the second part 22 composed of the first layer 20a and the second layer 20b of the parison 200. In this case, a mold 300 as shown in FIG. 13 may be used. The mold 300 has a longitudinal axis direction x3, a radial direction y3, and a circumferential direction z3, and has a lumen 305 that extends in the longitudinal axis direction x3 and into which the parison 200 is inserted. A portion of the parison 200 in the longitudinal axis direction x2 may be positioned within the lumen 305 of the mold 300. The stretching of the parison 200 may be performed by blow molding the parison 200, or by biaxially stretching the parison 200.

The mold 300 may include, in the longitudinal axis direction x3: a mold straight tubular part 300C for forming the straight tubular part 27 of the balloon 20; two mold tapered parts 300T disposed on both sides of the mold straight tubular part 300C for forming the tapered parts of the balloon 20; and two mold sleeve parts 300S disposed on sides farther from the mold straight tubular part 300C than the mold tapered parts 300T for forming the sleeve parts of the balloon 20. Accordingly, the straight tubular part 27 of the balloon 20 can be formed by the mold straight tubular part 300C, the proximal tapered part 26 and the distal tapered part 28 can be formed by the mold tapered parts 300T, and the proximal sleeve part 25 and the distal sleeve part 29 can be formed by the mold sleeve parts 300S.

The mold 300 may be composed of a single member or of multiple members. As shown in FIG. 13, the mold 300 may be formed by connecting a plurality of mold components in the longitudinal axis direction x3. For example, the mold straight tubular part 300C, the mold tapered part 300T, and the mold sleeve part 300S may be separate mold components that are connected to one another in the longitudinal axis direction x3. The mold 300 may also be configured to be separable in the radial direction y, which facilitates the insertion of the parison 200 into the mold cavity 305. As shown in FIG. 13, each mold component may be joined by engaging with an adjacent mold component. Alternatively, although not shown in the figures, each adjacent mold component may be provided with a magnet and joined together by magnetic attraction.

As shown in FIG. 14, the mold cavity 305 of the mold 300 may be formed in a substantially circular shape. By placing the parison 200 in such a mold cavity 305 and introducing a fluid into the inner cavity 205 of the parison 200 to perform biaxial stretching, a balloon 20 having a thickness variation ratio within a predetermined range can be manufactured.

The mold 300 may be made of a metal, and the metal may be iron, copper, aluminum, or an alloy thereof. For example, stainless steel may be used as an iron alloy, brass may be used as a copper alloy, and duralumin may be used as an aluminum alloy. The mold 300 may be made of stainless steel from the standpoint of having sufficient strength and ease of processing.

### 2. Balloon Catheter

A balloon catheter 10 according to one or more embodiments of the present invention includes the above-described balloon 20 for a balloon catheter. As described in the section “1. Balloon for balloon catheter,” the balloon 20 is connected to a distal end portion of a shaft 30, as shown in FIG. 1.

FIG. 1 illustrates a so-called rapid-exchange type balloon catheter 10, which has a guidewire port 61 located midway between the distal and proximal sides of the shaft 30 and has an inner shaft 60 that functions as a guidewire lumen from the guidewire port 61 to the distal side of the shaft 30. The balloon catheter 10 may have a distal shaft 31 and a proximal shaft 32, with the distal shaft 31 and the proximal shaft 32 being separate members. The proximal end part of the distal shaft 31 may be connected to the distal end part of the proximal shaft 32, thereby forming the shaft 30 that extends from the balloon 20 to the proximal end part of the balloon catheter 10. Alternatively, a single shaft 30 may extend from the balloon 20 to the proximal end part of the balloon catheter 10, or the distal shaft 31 and the proximal shaft 32 may each consist of multiple tube members.

The shaft 30 may have a fluid flow path and a guidewire lumen inside. To configure the shaft 30 to have the internal fluid flow path and the guidewire lumen, for example, the inner shaft 60 located inside the shaft 30 may function as the guidewire lumen, and the space between the shaft 30 and the inner shaft 60 may function as the fluid flow path. In such a configuration, the inner shaft 60 may extend from the distal end of the shaft 30 and pass through the balloon 20, the distal side of the balloon 20 may be connected to the inner shaft 60, and the proximal side of the balloon 2 may be connected to the shaft 30.

The shaft 30 may be composed of resin, metal, or a combination of resin and metal. By using resin as the material for the shaft, flexibility and elasticity can be more easily imparted to the shaft 30. By using metal as the material for the shaft 30, the delivering performance of the balloon catheter 10 can be improved. Examples of resin used for the shaft 30 include polyamide-based resin, polyester-based resin, polyurethane-based resin, polyolefin-based resin, fluorine-based resin, polyvinyl chloride-based resin, silicone-based resin, natural rubber, and synthetic rubber. Any one of these may be used alone, or two or more may be used in combination. Examples of metal used for the shaft 30, include stainless steel such as SUS 304 and SUS 316, platinum, nickel, cobalt, chromium, titanium, tungsten, gold, Ni—Ti alloys, Co—Cr alloys, or combinations thereof. When the shaft 30 is composed of the distal shaft 31 and proximal shaft 32 as separate members, the distal shaft 31 may be, for example, made of resin, and the proximal shaft 32 may be made of metal. The shaft 30 may also have a layered structure using different materials or the same material.

The balloon 20 and the shaft 30 may be joined by adhesive bonding, welding, or by attaching a ring-shaped member at the point where the end of the balloon 20 and the shaft 30 overlap to swage them. Of these, the balloon 20 and the shaft 30 may be joined by welding. By welding the balloon 20 and the shaft 30, the bond between the balloon 20 and the shaft 30 is difficult to be released even when the balloon 20 is repeatedly inflated and deflated, easily increasing the strength of the bond between them.

The balloon catheter 10 may be provided with a tip member 70 at its distal end part. The tip member 70 may be provided at the distal end part of the balloon catheter 10 by being connected to the distal end part of the balloon 20 as a separate component from the inner shaft 60, or the inner shaft 60 extending distally beyond the distal end of the balloon 20 may function as the tip member 70.

A radiopaque marker 80 may be placed on the inner shaft 60 inside the balloon 20 at the location of the balloon 20 in the longitudinal axis direction x1, so that the position of the balloon 20 can be confirmed radiographically. The radiopaque marker 80 may be placed at a position corresponding to both ends of the straight tubular part 27 of the balloon 20, or may be placed at a position corresponding to the center of the straight tubular part 27 in the longitudinal axis direction x1.

A hub 40 may be provided at a proximal side of the shaft 30, and the hub 40 may be provided with a fluid inlet 50 that is connected to the flow channel of the fluid supplied to the interior of the balloon 20.

The shaft 30 and the hub 40 may be joined by, for example, adhesive bonding or welding. Of these, the shaft 30 and the hub 40 may be joined by adhesive bonding. The adhesive bonding of the shaft 30 and hub 40 can increase the bonding strength of the shaft 30 and hub 40 to increase durability of the balloon catheter 10 when the materials forming the shaft 30 and hub 40 are different, for example, in a case where the shaft 30 is made of material having high flexibility and the hub 40 is made of material having high stiffness.

Although not shown in the figures, one or more embodiments of the present invention are also applicable to a so-called over-the-wire type balloon catheter that has a guidewire lumen extending from the distal end to the proximal end of the shaft. In the case of the over-the-wire type, the inflation lumen and the guidewire lumen may extend to a hub positioned at the proximal side, and the proximal openings of each lumen may be provided in the hub having a bifurcated structure.

In the case of the rapid-exchange type catheter, the outer wall of the distal shaft 31 and/or the proximal shaft 32 may be coated as appropriate, or both the distal shaft 31 and the proximal shaft 32 may be coated. In the case of the over-the-wire type catheter, the outer wall of the outer shaft may be coated as appropriate.

The coating can be a hydrophilic or hydrophobic coating, depending on the purpose, and can be applied by dipping the shaft 30 into a hydrophilic or hydrophobic coating agent, applying a hydrophilic or hydrophobic coating agent to the outer wall of the shaft 30, or coating the outer wall of the shaft 30 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coat, diamond coat, diamond-like carbon (DLC) coating, ceramic coating, and substances with low surface free energy terminated with an alkyl group or a perfluoroalkyl group.

The present application claims priority based on Japanese Patent Application No. 2022-184269 filed on Nov. 17, 2022. All the contents described in Japanese Patent Application No. 2022-184269 filed on Nov. 17, 2022 are incorporated herein by reference.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### DESCRIPTION OF REFERENCE SIGNS

10: balloon catheter
20: balloon for balloon catheter
20a: first layer
20A: first-layer-rich portion
20b: second layer
20B: second-layer-rich portion
20C: centroid of outer edge of balloon
21: first part
22: second part
30: shaft
31: distal shaft
32: proximal shaft
40: hub
50: fluid inlet
60: inner shaft
61: guidewire port
70: tip member
80: marker
200: parison
200a: first layer of parison
200b: second layer of parison
201: first end of parison
202: second end of parison
205: lumen of parison
208: protrusion part of parison
210: thick portion
220: thin portion
230: medium-thickness portion
250: parison mold
251: first tubular member
252: second tubular member
253: third tubular member
300: mold
300C: mold straight tubular part
300S: mold sleeve part
300T: mold tapered part
305: lumen of mold

1. A balloon for a balloon catheter having a longitudinal axis direction, a radial direction, and a circumferential direction, comprising:
a first layer disposed over the entire 360° in the circumferential direction; and
a second layer composed of a material having a Shore D hardness higher than that of the first layer, wherein the balloon comprises: in a cross-section perpendicular to the longitudinal axis direction,
a first part comprising the first layer where the second layer is not disposed on an outer side of the first layer in the radial direction; and
a second part comprising the first layer and the second layer where the second layer is disposed on the outer side of the first layer in the radial direction, wherein the first part is disposed in the circumferential direction within a range of 90° or less out of the 360° in the circumferential direction.

2. The balloon for the balloon catheter according to claim 1, wherein the first part extends in the longitudinal axis direction.

3. The balloon for the balloon catheter according to claim 2, wherein the first part comprises a plurality of first parts and the second part comprises a plurality of second parts, wherein:
the first parts are disposed apart from each other in the circumferential direction; and
an outer peripheral length in the circumferential direction per one of the second parts is twice or more than an outer peripheral length in the circumferential direction per one of the first parts.

4. The balloon for the balloon catheter according to claim 3, wherein the second part comprises a portion P1 in which T1 is more than 50% of T and a portion P2 in which T2 is more than 50% of T, where the T1 is a thickness of the first layer in the second part in the radial direction, the T2 is a thickness of the second layer in the second part in the radial direction, and the T is a sum of the T1 and the T2.

5. The balloon for the balloon catheter according to claim 4, wherein the second part further comprises:
a first-layer-rich portion in which an outer peripheral length in the circumferential direction of the portion P1 is 15% or more of the outer peripheral length in the circumferential direction per one of the second parts; and
two or more second-layer-rich portions, each having an outer peripheral length in the circumferential direction of the portion P2 that is 15% or more of the outer peripheral length in the circumferential direction per one of the second parts, wherein the first-layer-rich portion is sandwiched between the second-layer-rich portions in the circumferential direction.

6. A balloon catheter comprising the balloon according to claim 1.
